(19) United States
(12) Patent Application Publication
(10) Pub. No.: US 2018/0268923 A1
(43) Pub. Date: Sep. 20, 2018

(54) BIOMETRIC CONFIRMATION OF SAMPLE INTEGRITY

(71) Applicant: ALERE INC., Waltham, MA (US)

(72) Inventor: Namal NAWANA, Waltham, MA (US)

(21) Appl. No.: 15/762,882

(22) PCT Filed: Sep. 23, 2016
(86) PCT No.: PCT/US16/53277

§ 371 Date: Mar. 23, 2018

(51) Int. Cl. G16H 10/40; G01N 33/94; G06K 9/00

(52) U.S. Cl. CPC G16H 10/40; G01N 30/7233; G06K 9/00087; G01N 33/94

(57) ABSTRACT

Provided herein are new methods and systems to remotely administer toxicology tests while still preserving the chain of custody. These tea,methods and systems include ensuring that the tested samples originate from the subject and that the sample has not been adulterated. Ensuring that the tested samples originate from the subject and have not been adulterated is based on obtaining a first data of a first sample and comparing it to a second data of a second sample from the same subject.

### CLAIM OF PRIORITY

This application claims the benefit of U.S. Provisional Patent Application Ser. No. 62/232,624, filed on Sep. 25, 2015, the entire contents of which are hereby incorporated by reference.

### FIELD OF THE INVENTION

The invention relates to confirming the integrity of a sample.

### BACKGROUND

Toxicology testing is performed to determine whether a subject has ingested a drug (or failed to ingest a required drug). To secure the chain of custody and to prevent adulteration of the sample, a subject must typically provide the sample at a laboratory or other site configured to provide toxicology testing services. Thus, toxicology tests have not been administered remotely of a laboratory or other devoted site.

What is needed are methods and systems to permit toxicology tests to be administered remotely while preserving the chain of custody and ensuring that the tested sample originates from the subject and that the sample has not been adulterated.

### BRIEF DESCRIPTION OF THE FIGURES

FIG. 1 illustrates a urine collection cup;

FIG. 2 illustrates a biological sensor of the urine collection cup of FIG. 1;

FIG. 3 illustrates steps of a method for method for securing a chain of custody of a urine sample and for determining the integrity of the urine sample using the collection cup of FIG. 1; and

FIG. 4 illustrates steps of a method for method for securing a chain of custody of a sample and for determining the integrity of the sample.

### DETAILED DESCRIPTION

An example of a method for securing a chain of custody of a sample and for determining the integrity of the sample is described.

An individual secures employment in the transportation industry. The employee's position requires toxicology testing at frequent intervals. Because the employee travels to remote locations, it is inconvenient and costly for the employee to visit a laboratory for the required toxicology testing. Accordingly, the employee participates in remote toxicology testing as follows.

The employee visits a toxicology testing laboratory. Under secure and supervised conditions, a urine sample, an oral fluid sample, and a cheek swab sample are obtained from the employee. A fingerprint scan is obtained using a high fidelity fingerprint scanner. In each case, the chain of custody is preserved. Each sample is tested for adulteration by confirming that (i) there are no denatured proteins present (these would indicate a urine sample that was not fresh, e.g., a sample that had been previously given and then stored), (ii) sample temperature is within an acceptable range of physiological temperature (variation from the acceptable range indicates a sample that is not fresh), and (iii) for the presence of adulterants, e.g., reagents such as masking agents or diuretics.

The urine sample is analyzed using liquid chromatograph—mass spectrometry (LC-MS) to obtain a urine profile data set including the presence and amount of each of a plurality of biological compounds and metabolites. The urine profile data set is compared to urine profiles obtained by LC-MS from many other individuals to identify a urine profile subset of biological compounds and metabolites (e.g., the presence and/or amount of each) more likely to be associated with the employee than another individual. The oral fluid sample is analyzed in the same way as the urine sample to identify an oral fluid profile subset of biological compounds and metabolites (e.g., the presence and/or amount of each) more likely to be associated with the employee than another individual and also to identify unique DNA sequences.

DNA present in each sample is analyzed using a high resolution DNA sequencer to identify respective unique DNA sequences. DNA sequences derived from the urine sample, oral fluid sample, and cheek swab are compared to confirm that (i) the DNA derives from the same individual and (ii) that DNA from more than one source, e.g., DNA from a second individual, is not present. Although the urine, oral fluid, and cheek swab samples have been obtained under controlled conditions, the confirmation that the same unique DNA sequences are present in each sample (and that other sequences are absent) provides additional confirmation that the samples originate from the employee and not from another individual.

The high fidelity fingerprint image is analyzed to identify a subset of features unique to the employee's fingerprint. Advantageously, the identified subset of features have the further property that they can reliably be discerned in a fingerprint image obtained of the same digit using a lower fidelity scanner.

All of the data are stored in an electronic record of the employee. The employee's medical record is in electronic communication with a computer program that automates the remote toxicology testing (“Toxicology Program”).

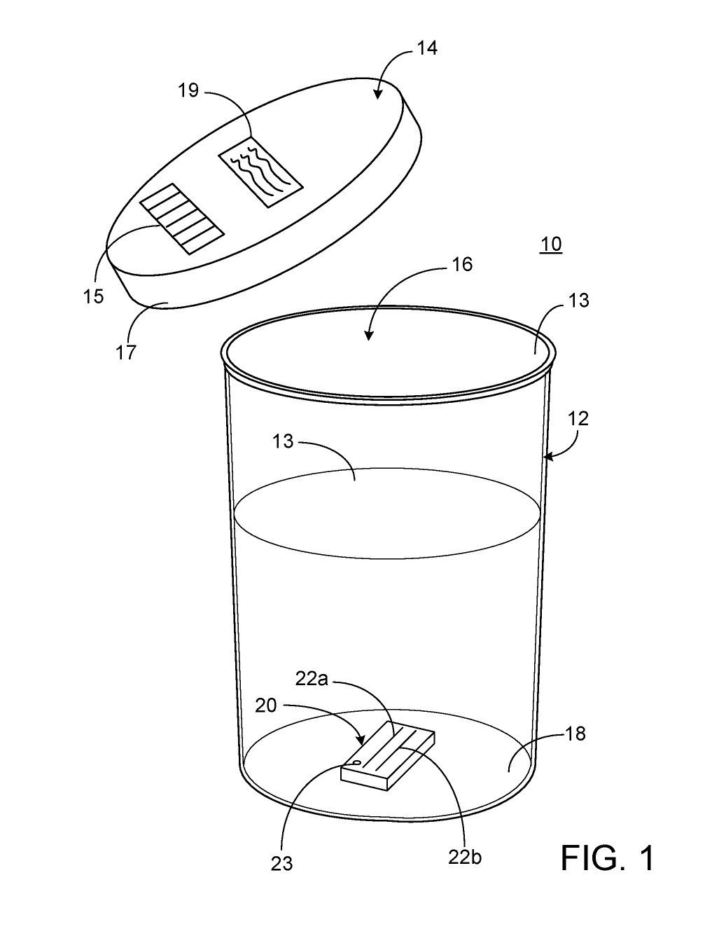

The employee is given a kit including (i) urine collection cups 10 (FIG. 1) and (ii) oral toxicology sampling devices. The employee leaves the facility and begins duties associated with employment.

Two weeks after leaving the toxicology laboratory, the Toxicology Program determines that the employee must take a toxicology test. However, before alerting the employee, the Toxicology Program checks the GPS signal of the employee's cell phone and determines that the employee is in motion (e.g., driving) and therefore is not in a position to administer the test. Accordingly, the Toxicology Program stays the alert for a period of time. This information is stored in the employee's electronic record. After the elapsed time, the Toxicology Program determines from the employee's cell phone GPS signal that the employee is not in motion and issues an alert to the cell phone.

An app on the employee's cell phone receives an alert signal from the Toxicology Program and alerts the employee that a toxicology test must be performed. The alert is given without prior notice to the employee and must be acknowledged by the employee within a predetermined time. The employee timely acknowledges the alert by pressing the same digit used to provide the fingerprint in the toxicology lab to the fingerprint scanner of the cell phone. The cell phone fingerprint scanner acquires a low fidelity fingerprint image and transmits the image to a remote processor in communication with the Toxicology Program. The Toxicology Program compares features of the low resolution fingerprint image to the identified subset of features in the high fidelity image acquired at the laboratory. After confirming with a high level of confidence that the images are of the same digit, the Toxicology Program sends a signal to the cell phone which in turn sends a signal to a collection cup 10 activating a toxicology test. The employee acknowledgement of the alert and result of the fingerprint scan are also stored in the electronic record of the employee.

The employee removes lid 14 of cup 10 and urinates into collection void 16 of cup 10 and activates the test by sealing cup 10 with lid 14. Cup 10 determines that lid 14 had been removed (and had not previously been removed) and urine deposited into the void 16 of cup 10 within a predetermined time of the alert. (If the lid 14 had been removed or urine deposited outside of this time limit, the test would be declared void. Cup 10 would send this information to the cell phone which would transmit the information to the Toxicology Program for storage in the employee's electronic record. This reduces the opportunity for the use of a pre-prepared and stored urine sample.). Accordingly, cup 10 initiates toxicology testing of the urine sample.

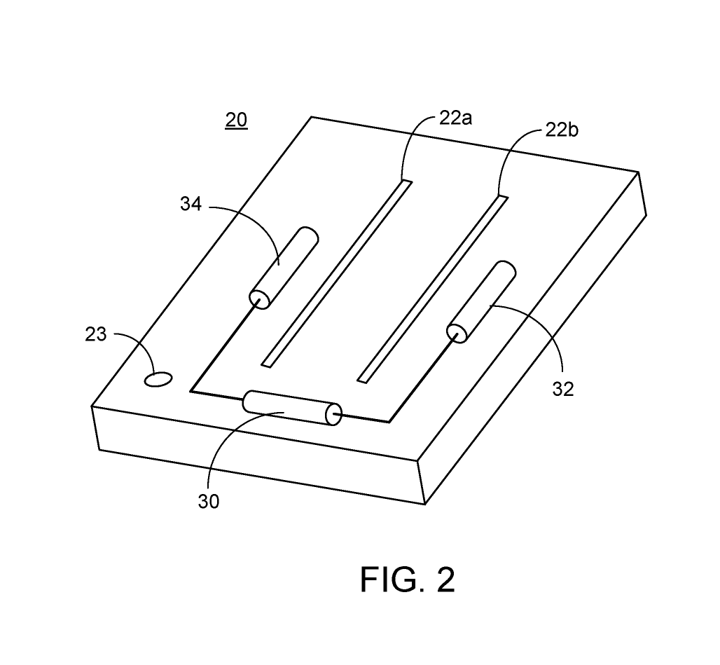

Sensors 22a,22b of cup 10 (see also FIG. 2) analyze the urine sample for the presence of drugs, drug metabolites, adulterants, and for biological compounds and metabolites associated with the urine profile subset determined from the urine obtained from the sample given at the toxicology testing laboratory. Sensors 22a,22b of cup 10 also analyze the urine sample for denatured proteins or other markers indicating that the urine sample had been stored for a period of time. DNA detector 23 of cup 10 analyzes the sample for the presence of the unique DNA sequences determined from the DNA obtained at the toxicology testing laboratory. Cup 10 measures the temperature of the urine. Cup 10 also analyzes the urine for the presence of denatured proteins associated with storage of the sample.

Collection cup 10 transmits data from the analyses to the employee's cell phone which transmits the data directly to the employee's electronic record. Cup 10 also confirms that the urine test is initiated within the predetermined time limit after the alert and that the lid 14 had been removed in performing the test but had not previously been removed. This information also transmitted to the employee's cell phone and then to the electronic record.

The Toxicology Program analyzes the urine toxicology data obtained by cup 10 and confirms the absence of drugs, drug metabolites and adulterants. The Toxicology Program then analyzes the urine profile data obtained by cup 10 and compares the data to the urine profile subset obtained from the urine sample given at the toxicology testing laboratory. The Toxicology Program determines that the urine temperature as measured by cup 10 is within the acceptable range. The Toxicology Program confirms with a high level of confidence that the biological compounds and metabolites of the urine analyzed by cup 10 match the urine profile subset in the employee's electronic record. The Toxicology Program also analyzes the data obtained by DNA detector 23 of cup 10 and confirms the presence of target DNA sequences identified based on the DNA sequence data obtained from the urine sample provided at the toxicology testing laboratory. Based on the data received from cup 10, the Toxicology Program determines that (i) the urine sample was negative for drugs, drug metabolites, adulterants and masking agents, (ii) the urine sample was delivered within the predetermined time limit of the alert, (iii) the urine sample did not indicate that the sample had been previously delivered and then stored, and (iv) the urine sample originated with a high level of confidence from the employee. These data are stored in the employee's electronic record. A message indicating a successful test is transmitted to the employee and the employer.

Three days after administration of the first toxicology test, the Toxicology Program determines that the employee must take another toxicology test. Before alerting the employee, the Toxicology Program checks the GPS signal of the employee's cell phone and determines that the employee is in not in motion (e.g., not driving) and therefore may be in a position to administer the test. The Toxicology Program sends a signal to the employee's cell phone which alerts the employee as to the test. The employee acknowledges an alert with a finger print (which is transmitted to the employee record and verified as previously). However, the employee also indicates via the cell phone app that the employee is not in a discrete location that would permit administration of a urine test but is able to administer an oral fluid toxicology test. This information is transmitted to the Toxicology Program which alerts a toxicology staff member who activates a live interaction with the employee via the cell phone. The employee acknowledges the application and begins audio-visual communication with the staff member.

The staff member confirms that the employee has an oral fluid toxicology test and sends a signal to the cell phone which in turn sends a signal activating the toxicology test. The employee, in video view of the staff member, opens the sealed sample application member of the oral toxicology test and swabs the interior of the employee's mouth collecting fluid. The employee then seals the sample application member of the test. The oral toxicology test sends a signal to the cell phone confirming that (a) the test had not been opened or tampered with prior to activation by the cell phone and (b) that the sample application member had been resealed to prevent tampering with the test or with the oral fluid sample sealed within the test. This preserves the oral fluid sample in the event that a backup analysis of the sample is required. The staff member sends a signal to the Toxicology Program indicating that the employee administered the test properly and that the test has been resealed in view of the staff member.

The oral toxicology test analyzes the oral fluid sample for the presence of drugs, drug metabolites, adulterants, and for biological compounds and metabolites associated with the oral fluid profile subset determined from the oral fluid obtained from the sample given at the toxicology testing laboratory. The oral toxicology test transmits data from the analyses to the employee's cell phone which transmits the data directly to the employee's electronic record. The oral toxicology test also confirms that the oral fluid test is initiated within the predetermined time limit after the alert and this information is also transmitted to the electronic record. After a delay, the information is also transmitted to the employee's cell phone and communicated to the employee.

The Toxicology Program analyzes the oral toxicology data obtained by the oral toxicology test and confirms the absence of drugs, drug metabolites and adulterants. The Toxicology Program then analyzes the oral fluid profile data obtained by the oral toxicology test and compares the data to the oral fluid profile subset obtained from the oral fluid sample given at the toxicology testing laboratory. The Toxicology Program confirms with a high level of confidence that the biological compounds and metabolites of the oral fluid match the oral fluid profile subset in the employee's electronic record. Based on the data received from oral fluid toxicology test and from the staff member, the Toxicology Program determines that (i) the oral fluid sample was negative for drugs, drug metabolites, adulterants and masking agents, (ii) the oral fluid sample was delivered within the predetermined time limit of the alert, and (iii) the oral fluid sample originated with a high level of confidence from the employee. These data are stored in the employee's electronic record.

The Toxicology Program also transmits a signal to the employee's cell phone which generates an alert requesting that the employee to ship the sealed test to the toxicology laboratory. This request is made randomly made for a small percentage of negative tests and for each positive test. The employee delivers the test to a drop site (e.g., a mailbox) and confirms the delivery via the cell phone app which transmits the information to the employee electronic record. In this example, the test was negative for all drugs, metabolites and adulterants. When the test is received by the toxicology laboratory, the receipt of the test is entered into the employee electronic record along with data from the toxicology test indicating that the test had not been unsealed after administration or tampered with. In the event that the test had been a positive test, a toxicology staff member unseals the test and performs a backup test of the remaining oral fluid to confirm the positive result. The test is configured to store data indicating whether the test had been unsealed (or tampered with) after administration but prior to access via the toxicology laboratory. This ensures the chain of custody of the oral sample. By issuing ‘return test’ alerts in a small percentage of negative tests, the Toxicology Program makes compliance with this request more likely than if such alerts were only issued in cases of positive results.

Methods for securing a chain of custody of toxicology samples and for determining the integrity of the toxicology samples as described herein may be performed for subjects who must submit to toxicology testing on a recurring basis. For example, a subject may be a person who submits to toxicology testing in connection with the person's employment or with a court order. It would be inconvenient and costly for the subject to travel to a toxicology testing laboratory. In accordance with the method, toxicology tests are administered at remote locations, away from any testing facility, healthcare professional, or other professional. For example, the subject may provide a urine sample from within the residence (e.g., home) of the subject. The method permits the integrity of even remotely collected samples to be maintained and confirmed. For example, the method preserves the chain of custody of the samples and any toxicology data resulting from the toxicology testing so that the samples and toxicology data can be linked to the subject without the uncertainty that might result from tampering by an individual other than the subject and/or from mishandling of the sample. In addition, the method permits an employer or testing agency to determine and confirm that the subject (and not another individual) is the source of the sample and that the sample has not been tampered with (e.g., by the subject) so as to modify a result of the toxicology testing. The method thereby protects the subject by ensuring that the provided sample has not been tampered with or confused with a sample of another person. The method also protects a party such as the subject's employer by ensuring that the sample is from the subject and has not been tampered with.

With reference to FIG. 1, a urine collection cup 10 is described. Collection cup 10 is used for collecting a urine sample, preserving the chain of custody of the urine sample, and confirming the integrity of the urine sample. Collection cup 10 includes a body 12 and a lid 14. Body 12 includes a wall 13 and a bottom 18 defining a void 16 configured to retain a urine sample 13. Cup 10 also includes a processor 30, a power supply 32, and an antenna 34.

Lid 14 includes machine readable identifying indicia 15 and a tamper resistant seal 17 operable to indicate whether cup 10 has been tampered with once sealed. Tamper resistant seal 17 also operates to determine whether the lid 14 has been opened prior to pre-determined time. Tamper resistant seal communicates with processor 30, which stores data indicative of when and where lid 14 was removed or replaced. Exemplary cups for securing a chain of custody of samples (specimens) including identifying indicia are described in U.S. Pat. No. 9,018,015 (the “'015 Patent”). Such features may be utilized for cup 10. Lid 14 also includes a display screen 19 for displaying information concerning the operation and status of cup 10.

Also with reference to FIG. 2, the bottom 18 of cup 10 further includes a biological sensor 20 configured to perform a toxicology test on the urine sample 13 to determine toxicology data indicative of whether the subject has or has not used one or more drugs. To perform the toxicology test, the biological sensor includes sensors 22a,22b configured to determine toxicology data indicative of the presence and/or amount of one or more drugs or metabolites thereof. Sensors 22a,22b include immunological sensors capable of detecting biomolecules such as proteins and small molecules such as drugs and drug metabolites. Sensors 22a,22b also include electrochemical sensors capable of detecting pH, ions, and dissolved gases. Sensors 22a,22b are also configured to determine the presence of masking agents or other adulterating compounds that might interfere with the proper determination of the toxicology data. Sensors 22a,22b are also configured to determine biological compounds and metabolites (e.g., the presence and/or amount of each) of a urine profile more likely to be associated with a particular individual than with another individual.

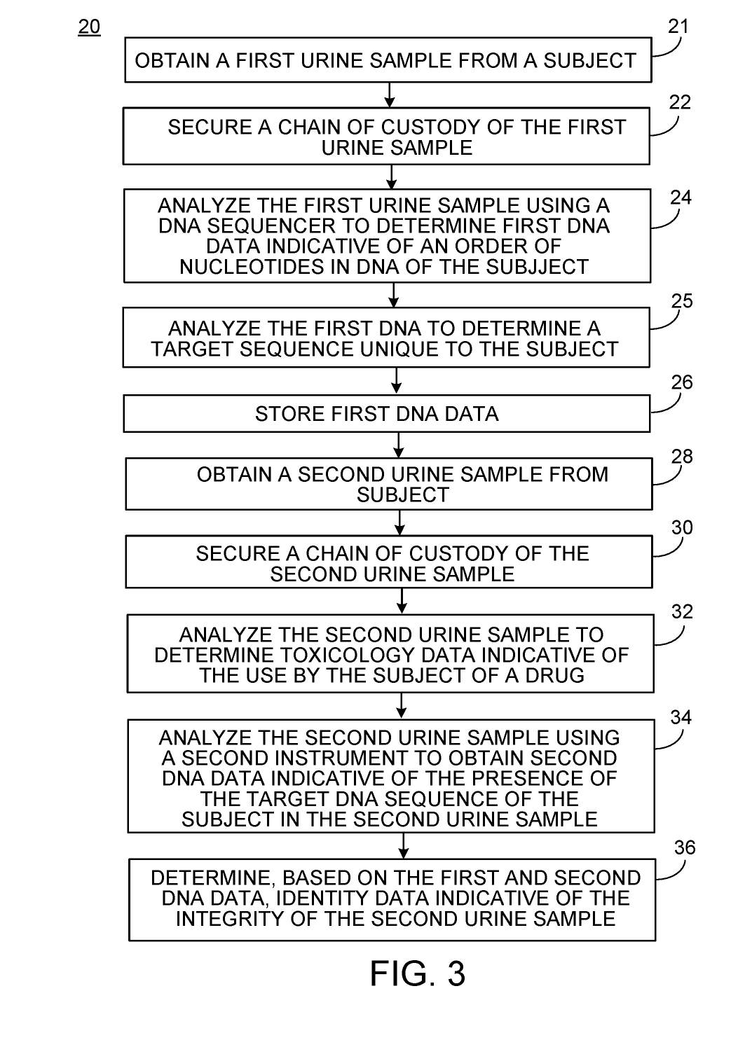

Biological sensor 20 also includes a DNA detector 23 for determining DNA data indicative of the presence of one or more target DNA sequences from the subject present in sample 13. In use, DNA detector 23 performs isothermal amplification of DNA present in sample 13. Exemplary methods and devices for determining the presence of one or more target DNA sequences using isothermal amplification are described in U.S. Pat. No. 9,057,097 (the “'097 Patent”) and in U.S. patent application Ser. No. 11/778,018 filed 14 Jul. 2007 (the “'018 Application”) and may be utilized for sensor 23. Processor 30 operates sensors 22a,22b and DNA detector 23 to determine the toxicology data and the DNA data. Antenna 34 permits processor 30 to communicate with display 19 of lid 14 and also to communicate the toxicology data and the DNA data to an external source such as a cell phone as discussed below with respect to FIG. 3.

With reference to FIG. 3, a method for securing a chain of custody of a urine sample and for determining the integrity of the urine sample is described. In step 21, a first urine sample, a first oral fluid sample, and a first cheek swab sample is obtained from a subject, e.g., a human. These samples are provided under controlled conditions to minimize the opportunity for tampering. Each sample is deposited into a container and sealed. In a step 22, a chain of custody of each of the urine, oral fluid, and cheek swab samples is secured to prevent tampering with or accidental mismanagement of the samples. Exemplary methods and systems for securing a chain of custody of samples (specimens) are described in the '015 Patent and may be utilized in steps 21 and 22 of method 21.

Each of the samples is tested for adulteration or other manipulation. For example the samples are tested for adulteration by confirming that (i) there are no denatured proteins present (these would indicate a sample that was not fresh, e.g., a sample that had been previously given and then stored), (ii) sample temperature is within an acceptable range of physiological temperature (variation from the acceptable range indicates a sample that is not fresh), and (iii) for the presence of adulterants, e.g., reagents such as masking agents or diuretics.

In a step 24, the samples are analyzed using a DNA sequencer to determine first DNA data indicative of an order of nucleotides in DNA each sample. In a step 25, the first DNA data are analyzed to identify one or more target sequences unique to the DNA present in the samples. By unique, it is meant that the presence of the one or more target sequences in a DNA sample can be used with a high level of confidence to identify the DNA sample as originating from the subject as opposed to originating from one or more other subjects. As used herein, the level of confidence is at least 95%, at least 99%, at least 99.9%, at least 99.99%, at least 99.999%, or at least 99.9999%. Advantageously, the DNA sequencer used to obtain the first DNA data has a high informing power and is able to determine an order of >1000 nucleotides, >10,000 nucleotides, or more than 100,000 nucleotides in a single run. In a step 26, the first DNA data and the target DNA sequences of each of the urine sample, the oral fluid sample and the cheek swab sample are stored, e.g., in encrypted form in an electronic record of the subject that is secured against unauthorized access.

The urine sample is analyzed using high resolution liquid chromatography-mass spectrometry (LC-MS) to obtain a urine profile data set including the presence and amount of each of a plurality of biological compounds and metabolites. The oral fluid sample is similarly analyzed to obtain a corresponding oral fluid profile data set. The urine profile data set is compared to urine profiles obtained from many other individuals to identify a urine profile subset of biological compounds and metabolites (e.g., the presence and/or amount of each) more likely to be associated with the employee than another individual. The oral fluid data set is compared in the sample way to oral fluid profiles obtained from many other individuals to identify an oral fluid profile subset of biological compounds and metabolites (e.g., the presence and/or amount of each) more likely, e.g., with a high level of confidence, to be associated with the employee than another individual. In addition, the urine and oral fluid data sets are compared (along with the corresponding subsets) to confirm that each sample originated from the same subject and also to confirm the absence of manipulation by the subject.

Each of the first urine sample, first oral fluid sample, and first cheek swab sample is a confirmed integrity sample. Each sample is obtained under controlled conditions that confirm the subject as the source of the sample and minimize the probability that the sample is tampered with either by the subject or by anyone else. For example, the each sample is typically provided under supervision in a laboratory by professional in the toxicology testing industry.

The first DNA data and the target DNA data derived from the urine, oral fluid, and cheek swab samples are compared to compared to confirm that (i) each DNA derive from the same individual (i.e., the subject) and (ii) that DNA from another source, e.g., DNA from a second individual, is not present. Because the urine, oral fluid, and cheek swab samples have been obtained under controlled conditions, the unique DNA sequences of the target DNA data are confirmed as originating from the employee and not from another individual. Accordingly, the first DNA data and target DNA data derived from each sample are confirmed integrity data. In addition, the urine profile data set, the urine profile subset, the oral fluid profile data set, and the urine profile subject are also confirmed integrity data.

In a step 28, a second urine sample is obtained from the subject at a location remote from a toxicology laboratory using collection cup 10. The collection cup 10 is sealed. In a step 30, a chain of custody of the second urine sample is secured to prevent tampering with or accidental mismanagement of the urine sample. Methods and systems for securing a chain of custody of samples (specimens) are described in the '015 Patent and may be utilized in step 30 of method 21.

In a step 32, the second urine sample is analyzed using sensors 22a,22b configured to determine toxicology data indicative of the presence and/or amount of one or more drugs or metabolites thereof. In addition, sensors 22a,22b determine the presence of masking agents or other adulterating compounds that might interfere with the proper determination of the toxicology data and biological compounds and metabolites of the urine profile subset of associated with the subject.

In a step 34, the second urine sample is analyzed using DNA detector 23 of biological sensor 20 of cup 10 to determine second DNA data indicative of the presence or absence in the second urine sample of the target DNA sequence of the subject.

In a step 36, identity data indicative of the integrity of the second urine sample is determined based upon the first and second DNA data. Alternatively, or in addition, the identity data indicative of the integrity of the second urine sample is determined based upon data indicative of the target sequences and the second DNA data. Alternatively or in addition, the identity data are determined based upon data indicative of the urine profile subset and the biological compounds and metabolites of the urine profile subset measured by cup 10. The identity data are indicative of at least one of whether (a) the subject is the source of the second sample (b) the second sample comprises material derived from a different subject and (c) an apparent amount of a drug or metabolite thereof in the sample has been modified as compared to the actual amount present in the sample.

Sensors 22a,22b have a lower informing power than LC-MS. However, sensors 22a,22b are capable of determining the urine profile subset identified using the higher informing power of LC-MS. Advantageously, therefore, the higher informing power (and expensive) LC-MS may be run only once to identify a urine profile associated with a subject. Subsequently, the presence of the compounds of the urine profile subset are determined using a lower informing power (and less expensive) instrument (e.g., sensors 22a,22b).

DNA detector 23 has a lower informing power than the DNA sequencer used to obtain the first DNA data of step 24 of method 20. However, DNA detector 23 is capable of detecting the target DNA sequences identified using the higher informing power of a sequencing instrument in step 24. For example, DNA detector 23 may be capable of determining the presence and amount of up to five target DNA sequences each of 100 base pairs (bps) in length or less. As discussed above, the DNA sequencer used to obtain the first DNA data operates to determine an order of many more nucleotides in a single run. Advantageously, therefore, the higher informing power (and expensive) instrument may be run only once to identify one or more target sequences associated with a subject. Subsequently, the presence of the target sequences may be determined using a lower informing power (and less expensive) instrument (e.g., DNA detector 23) to obtain the second DNA data.

While steps 28, 30, 32, 34, and 36 of method 20 have been described in relation to a urine sample of a subject, other samples may be used. For example, an oral fluid sample or a blood sample may be used in addition to or as an alternative to a urine sample.

While method 20 has been described in relation to DNA data and LC-MS data other data indicative of a physiological or physical property of a user may be used. For example, first data indicative of a structure of a subject's skin, e.g., a fingerprint, may be obtained using a first system having a high informing power or fidelity. The first data are analyzed to determine target data indicative of a subset of the first data unique to the individual. Subsequently, a second system having lower informing power or fidelity than the first system may be used to determine the presence of the target data in a skin sample (e.g., an exposed digit) proffered by a subject in connection with a toxicology test. Advantageously, the second system may a disposable or reusable but field-deployable system of lower cost than the first system. The second system, however, reliably determines the presence or absence of the target data the skin sample so as to permit the determination with a high level of confidence of whether the skin sample is of the subject or of another origin, e.g., of another individual. The second system may be configured to obtain toxicology data non-invasively using the same (or through the same) skin sample proffered by the subject in connection with the toxicology test.

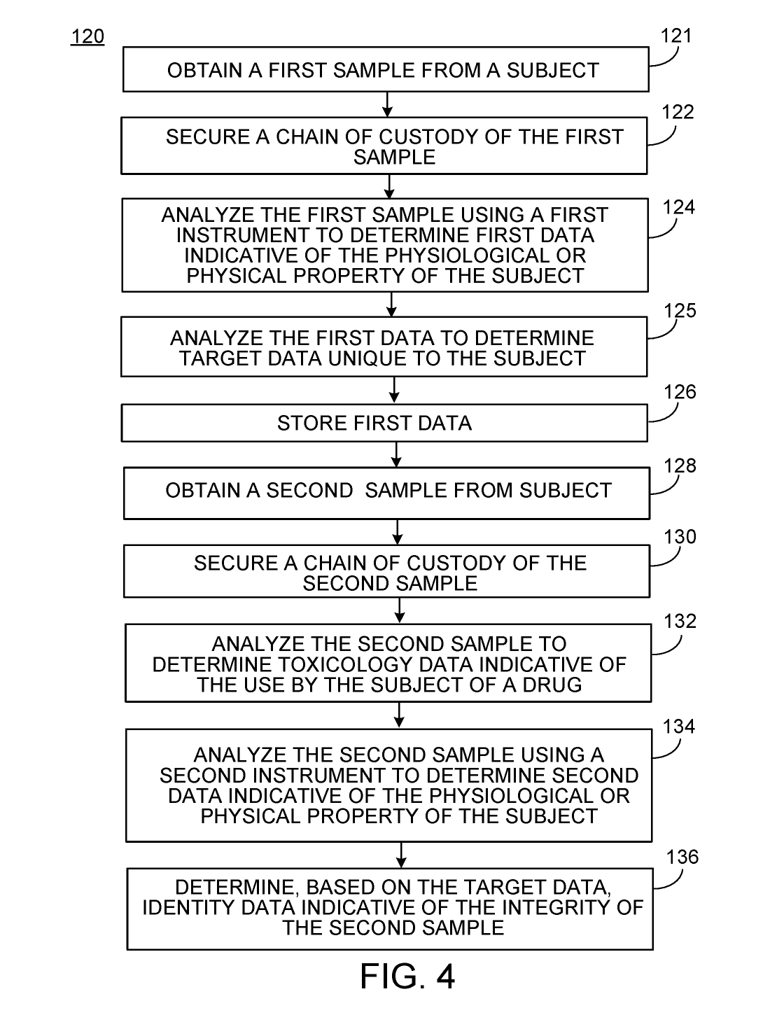

With reference to FIG. 4, a method 120 for securing a chain of custody of a sample and for determining the integrity of the sample is described. In step 121, a first sample is obtained from a subject, e.g., a human. The first sample is suitable for analysis to determine data indicative of a physiological or physical property of the subject. For example, the first sample may be a liquid from the subject (e.g., urine, blood, saliva, or tears) or a physical sample (e.g., a tissue or hair sample). Alternatively, the first sample may be an image sample obtained using, for example, electromagnetic (e.g., optical), ultrasound, or capacitive imaging. Examples of image samples include images of features of skin including skin of a digit of a human, as a fingerprint of the human. In a step 122, a chain of custody of the first sample is secured to prevent tampering with or accidental mismanagement of the sample.

In a step 124, the first sample is analyzed using a first instrument to determine first data indicative of a physiological or physical property of the subject. For example, the first instrument may be configured to obtain first data indicative of an ordering of nucleotides in DNA of the subject. The first instrument may be configured to obtain first data indicative of multiple constituents of a liquid or solid sample of the subject. For example, the first data may be indicative of the presence and amount of each of multiple microorganisms, e.g., bacteria, viruses, and/or fungi, present in the sample. The first instrument may be configured to obtain first data indicative of features of an image of the subject e.g., patterns and features of a fingerprint of the subject.

In a step 125, the first data are analyzed to identify one or more target data sets unique to the subject. By unique, it is meant that the presence of the one or more target data sets a set of data obtained from a sample can be used to identify the sample as originating from the subject as opposed to originating from one or more other subjects with a high level of confidence. Advantageously, the instrument used to obtain the first data has a high informing power or fidelity and thus is capable of producing data with high resolution, number of measurement channels, high precision and accuracy, and/or low noise.

Each of the first sample, first data, and target data are confirmed integrity data. The first sample is obtained under controlled conditions that confirm the subject as the source of the first sample and minimize the probability that the urine sample is tampered with either by the subject or by anyone else. For example, the urine sample may be provided under supervision in a laboratory by professional in the toxicology testing industry.

In a step 126, the first data are stored, e.g., in encrypted form in a database secured against unauthorized access. Alternatively, or in addition, the target data may be stored, e.g., in encrypted form in a database secured against unauthorized access

In a step 128, a second sample is obtained from the subject. In a step 130, a chain of custody of the second sample is secured to prevent tampering with or accidental mismanagement of the sample. Methods and systems for securing a chain of custody of samples (specimens) are described in the '015 Patent and may be utilized in step 130 of method 121.

In a step 132, the second sample is analyzed to determine toxicology data indicative of the presence and/or amount of one or more drugs or metabolites thereof. In addition, the second sample may be analyzed to determine the presence of masking agents or other adulterating compounds that might interfere with the proper determination of the toxicology data.

In a step 134, the second sample is analyzed using a second instrument to determine second data indicative of the physical or physiological property of the subject. In a step 136, identity data indicative of the integrity of the second sample is determined based upon the first and second data. In addition, or in alternative, identity data indicative of the integrity of the second sample may be determined based upon the first target data and second data. The identity data are indicative of at least one of whether (a) the subject is the source of the second sample (b) the second sample comprises material derived from a different subject and (c) an apparent amount of a drug or metabolite thereof in the sample has been modified as compared to the actual amount present in the sample

The instrument used to obtain the second data has a lower informing power or fidelity than the instrument used to obtain the first data of step 124 of method 120. For example, the second data may be of lower resolution, smaller number of measurement channels, lower precision or accuracy, and/or higher noise than the first data. Advantageously, therefore, the higher informing power (and expensive) instrument of step 124 may be run only once to identify one or more target sequences associated with a subject. Subsequently, a lower informing power (and less expensive) instrument may be used to obtain the second data of step 134.

Each patent and patent application referenced herein is incorporated herein in its entirety.

1. A method, comprising:
obtaining first data of a first sample obtained from a mammalian subject, the first data being indicative of at least one of a physiological or physical property of the subject;
subsequently, obtaining second data of a second sample obtained from the subject, the second data being indicative of the at least one physiological or physical property of the subject, an informing power of the first data set being greater than an informing power of the second data set; and
based upon a comparison of the first and second data, determining at least one of whether (a) the subject is the source of the second sample (b) the second sample comprises material derived from a different subject and (c) an apparent amount of a drug or metabolite thereof in the sample has been modified as compared to the actual amount present in the sample.

2. The method of claim 1, wherein obtaining the first data comprises analyzing the first sample with a first instrument and obtaining the second data comprises analyzing the second sample with a second instrument, the second instrument being capable of a lower informing power than the first instrument.

3. The method of claim 1, further comprising obtaining, concurrently with the step of obtaining the second data, toxicology data indicative of the presence of the drug or degradation product thereof in the second sample.

4. The method of claim 1, further comprising transmitting the second data to a remote site.

5. The method of claim 1, wherein the step of obtaining second data comprising introducing a liquid sample from the subject to a container figured to preserve a chain of custody of the second sample.

6. The method of claim 5, wherein the step of obtaining second data is performed using a biological sensor in contact with the liquid sample in the container.

7. The method of claim 5, further comprising obtaining toxicology data indicative of the presence of the drug or degradation product thereof in the second sample by using a biological sensor in contact with the liquid sample in the container.

8. The method of claim 1 wherein the first and second data are indicative of an appearance of a skin of the subject.

9. The method of claim 8, wherein the first and second data are indicative of a fingerprint of a user.

10. The method of claim 8 or 9, further comprising obtaining, concurrently with the step of obtaining the second data, toxicology data indicative of the presence of the drug or degradation product thereof in the second sample.

11. The method of claim 10, wherein the step of obtaining toxicology data is performed non-invasively.

12. (canceled)

13. (canceled)

14. A system, comprising:
a database comprising confirmed integrity data of the subject, the confirmed integrity data being indicative of the at least one physical or physiological characteristic of a first subject;
a sensor configured to receive a sample from a second subject and analyze the sample to obtain toxicology data indicative of the presence of one or more drugs or metabolites thereof;
a sensor configured to obtain integrity data from the second subject, the integrity data being indicative of at least one physical or physiological characteristic of the subject and the sensor configured to obtain the integrity data having a lower informing power than a sensor used to obtain the confirmed integrity data;
a processor configured to determine, based on the confirmed integrity data and the integrity data whether the second subject is the first subject.

15. (canceled)

16. A method for remote toxicology testing, comprising:
initiating video communication between a first person at a first video display and a second person having a second video display wherein the first person and the second person are located remotely from one another; and
receiving, at the first video display, video data indicative of a self-administration by the second person of a toxicology test.

17. The method of claim 16, further comprising transmitting from the toxicology test a signal indicative of an operation of the toxicology test and receiving the signal at the second video display.

18. The method of claim 17, wherein the second video display is a display of a mobile phone associated with the second person.

19. The method of claim 16 further comprising receiving, at a computer system remote from the second video display the signal indicative of the operation of the toxicology test.

20. The method of claim 16, wherein the signal is indicative of at least one of (i) at time at which a sample application member of the toxicology test was unsealed, (ii) when a sample application member of the toxicology test was first unsealed or whether the toxicology test had been tampered with, (iii) a time at which a sample application member of the toxicology test was resealed and (iv) a result of the toxicology test.

21. The method of claim 16 further comprising, receiving, from a mobile device of the second person, a signal indicative of the availability of the second person for self-administration of an oral fluid toxicology test.

22. The method of claim 16 further comprising, receiving, from a mobile device of the second person, a signal indicative of the unavailability of the second person for self-administration of a toxicology test.

23. The method of claim 22, wherein the signal indicative of the unavailability of the second person is a GPS signal indicative of at least one of a position or motion of the

24. (canceled)

25. (canceled)

26. (canceled)

27. (canceled)

28. (canceled)
